(19) United States
(12) Patent Application Publication
(10) Pub. No.: US 2025/0178895 A1
(43) Pub. Date: Jun. 5, 2025

(54) VERSATILE AND FLEXIBLE, ENVIRONMENTALLY FRIENDLY AND ECONOMICALLY VIABLE PROCESS FOR CONVERTING SOUR NATURAL GAS TO SWEET NATURAL GAS, GREEN HYDROGEN AND CARBON DISULFIDE

(71) Applicant: B.G. NEGEV TECHNOLOGIES AND APPLICATIONS LTD., AT BEN-GURION UNIVERSITY, Beer Sheva (IL)

(72) Inventors: Mordechay HERSKOWITZ, Hadera (IL); Tomy HOS, Beer-Sheva (IL); Miron LANDAU, Beer-Sheva (IL); Roxana VIDRUK NEHEMYA, Beer-Sheva (IL)

(21) Appl. No.: 18/841,144

(22) PCT Filed: Feb. 23, 2023
(86) PCT No.: PCT/IL2023/050185

§ 371 Date: Aug. 23, 2024

Related U.S. Application Data
(60) Provisional application No. 63/314,488, filed on Feb. 28, 2022.

(51) Int. Cl. C01B 3/40; B01D 53/00; B01D 53/22; B01J 8/04; B01J 21/04; B01J 23/00; B01J 23/28; B01J 23/86; B01J 37/02; B01J 37/03; B01J 37/04; B01J 37/20; C01B 3/48; C01B 3/50; C07C 1/12

(52) U.S. Cl. CPC C01B 3/40; B01D 53/002; B01D 53/226; B01D 53/229; B01J 8/0492; B01J 8/0496; B01J 21/04; B01J 23/005; B01J 23/28; B01J 23/866; B01J 23/868; B01J 37/0201; B01J 37/036; B01J 37/04; B01J 37/20; C01B 3/48; C01B 3/501; C01B 3/506; C07C 1/12; B01D 2256/16; B01D 2257/304; B01D 2257/7025; C01B 2203/0238; C01B 2203/0283; C01B 2203/0405; C01B 2203/046; C01B 2203/048; C01B 2203/0485; C01B 2203/062; C01B 2203/0827; C01B 2203/1011; C01B 2203/1082; C01B 2203/1241; C01B 2203/148

(57) ABSTRACT

A process for preparing hydrogen by a catalytic conversion of sour natural gas, including feeding sour natural gas and one or more H2S recycled streams, optionally mixed with fresh CO2, to a reformer reactor packed with a catalyst activated in-situ by sulfidation. An apparatus for carrying out the process, to convert sour natural gas to sweet natural gas, hydrogen and carbon disulfide, and catalysts that can be used in the process, are also disclosed.

### CROSS-REFERENCE TO RELATED APPLICATIONS

This application is a National Stage application of International Patent Application No. PCT/IL2023/050185, filed on Feb. 23, 2023, which claims priority to U.S. Provisional Patent Application No. 63/314,488, filed on Feb. 28, 2022, each of which is hereby incorporated by reference in its entirety.

### TECHNICAL FIELD

The present disclosure relates to a versatile and flexible, environmentally friendly and economically viable process for converting sour natural gas to sweet natural gas, green hydrogen and carbon disulfide.

### BACKGROUND

Natural gas is classified according to its CO₂ and H₂S content: sweet natural gas that contains <2% CO₂ and <4 ppm H₂S used without further treatment and sour natural gas that does not meet the sweet natural gas criteria (Richard W. Baker and Kaaeid Lokhandwala, “Natural Gas Processing with Membranes: An Overview”, Ind. Eng. Chem. Res. 2008, 47, 2109-2121). There are various levels of sour natural gas, as listed in Table A. The very sour natural gas accounts for a relatively high percentage of the total natural gas available. One of the grand challenges in utilization of this large resource is to remove H₂S from such highly sour natural gas streams to enable their applications in the energy and chemical sectors. Therefore, there is a high incentive to develop a novel process that removes H₂S and CO₂ and produces sweet natural gas, hydrogen and carbon disulfide.

TABLE A

| | 1-15 mol % H₂S | >15 mol % H₂S |
|---|---|---|
| >15 mol % CO₂ | 0.1% | 4% |
| 0-15 mol % CO₂ | 30% | 0.9% |

Available world sour gas reserves (% of total gas) based on the levels of CO₂ and H₂S concentration (mol %). Adopted from William Taifan and Jonas Baltrusaitis, “Minireview: direct catalytic conversion of sour natural gas (CH₄+H₂S+CO₂) components to high value chemicals and fuels”, Catal. Sci. Technol., 2017, 7, 2919

The commercial technology currently employed to convert sour to sweet natural gas consists mainly of the acid gas removal unit to selectively strip off H₂S and CO₂ (amine-based absorption is frequently used) and sulfur recovery unit (SRU) where H₂S in the H₂S-rich stream reacts by the well-known Claus process to produce sulfur as depicted in FIG. 1 (Mansi S. Shah, Michael Tsapatsis and J. Ilja Siepmann, “Hydrogen Sulfide Capture: From Absorption in Polar Liquids to Oxide, Zeolite, and Metal-Organic Framework Adsorbents and Membranes”, Chem. Rev. 2017, 117, 9755-9803). The process is not economical at high H₂S concentration, small scale and when the price of sulfur is low, but it is considered as an environmentally acceptable manner to dispose of H₂S.

U.S. Pat. No. 10,759,722 relates to hydrogen sulfide methane reformation, showing that sour natural gas can be upgraded in a DHA reactor (dehydroaromatization) to produce liquid aromatic hydrocarbons and CS₂. Exemplified feed compositions have relatively low H₂S content.

Although hydrogen sulfide methane reformation offers a promising route to produce clean hydrogen while simultaneously removing hydrogen sulfide and eliminating the need for Claus unit, very little has been published on this topic (A. L. Martínez-Salazar, J. A. Melo-Banda, M. A. Coronel-García, Pedro M. García-Vite, Iris Martínez-Salazar, and J. M. Domínguez-Esquivel, “Technoeconomic analysis of hydrogen production via hydrogen sulfide methane reformation”, International Journal of Hydrogen Energy 44 (24), 2019, 12296-12302) with no real leads to commercial processes. Furthermore, a very high H2S/CH4 ratio at feed (>8) is usually used to avoid potential carbon deposits (Cunping Huang and T. Ali, “Liquid hydrogen production via hydrogen sulfide methane reformation”, Journal of power sources 175(1), 2008, 464-472), which will cause catalyst deactivation.

### SUMMARY

We have now developed an elegant process design that reacts methane with H₂S in sour or ultra-sour natural gas (possibly in the presence of CO₂) to produce hydrogen, CS₂ and sweet natural gas. That is, the feed to the reformer reactor consists of CH₄, H₂S and possibly CO₂; with the aid of selected catalysts capable of advancing both reactions (H₂S reforming and dry reforming), and under a set of suitable conditions, sour natural gas with high H₂S levels can be converted to sweet natural gas, hydrogen and carbon disulfide, as shown by reactions R1 and R2 below, achieving not less than 25% conversion, e.g., ≥30%, and even ≥40% H2S conversion (40-50%). As to the products obtained, the hydrogen can be further reacted with CO₂ to produce green liquid fuels and chemicals (Herskowitz, Mordechay and Hos, Tomy, “Novel, highly efficient eco-friendly processes for converting CO₂ or CO-rich streams to liquid fuels and chemicals”, U.S. Pat. No. 10,865,107 (2020)). CS₂ is a valuable material that is more desirable than elemental sulfur as a feedstock to produce chemicals.

Hydrogen sulfide methane reformation is shown by reaction R1:

CH₄+2H₂S→CS₂+4H₂ ΔH_{298K}=233 KJ/mole R1.

Since CO₂ may be a component of the feed, dry reforming may take place in parallel:

CH₄+CO₂→2CO+2H₂ ΔH_{298K}=247 KJ/mole R2.

and reverse water gas shift (RWGS) in series:

H₂+CO₂→CO+H₂O ΔH_{298K}=41 KJ/mole R3.

Since all reactions are reversible and endothermic, the product composition at thermodynamic equilibrium varies significantly over range of temperatures, pressure and feed composition. The equilibrium plot for reforming hydrogen sulfide and carbon dioxide with methane by Gibbs free energy minimization simulation indicates that temperature and pressure affect the equilibrium significantly, as shown in FIG. 2. Therefore, low pressure and high temperatures are necessary to obtain high H₂S conversion.

Accordingly, one aspect of the invention is a process for preparing hydrogen by catalytic conversion of sour natural gas, comprising feeding sour natural gas mixed with one or more H₂S recycled streams (which may contain also CH₄, H₂ and CO₂), and optionally fresh CO₂ to a reformer reactor packed with a catalyst, for example, a catalyst activated in-situ by sulfidation (selected catalysts are described in detail below).

Feed streams, with compositions suitable to afford hydrogen in industrially acceptable quantities, comprise from 50 to 90 vol % methane, not less than 10 vol % H₂S, e.g., for example from 10 to 40 vol % H₂S, and 0 to 40 vol % CO₂. The process is well suited to convert feed streams with >12 vol % H₂S, e.g., >15 vol % H₂S, for example, from 15 to 35 vol % H₂S.

The catalytic conversion of sour natural gas takes place over the catalyst in the reformer reactor under the following conditions: temperature in the range from 800 to 950° C., e.g., up to 900° C.; WHSVH2S in the range of 0.5-5 h⁻¹ at a total pressure of 1-3 atm.

The effluent from the reactor is passed through a separation system consisting of several units; unreacted feed material, namely, H₂S-containing streams, are collected at several points and are recycled to the reformer reactor, whereas CO₂ can be produced downstream and may be either directed to the reformer reactor, or even better, used as a feed component in a plant where CO₂ and hydrogen-the key product of the process-are converted into liquid hydrocarbons, e.g., as described in U.S. Pat. No. 10,865,107.

Separation of unreacted H₂S from the effluent of the reformer reactor, and separation of the effluent into a liquid stream consisting of the CS₂ by-product and the (CH₄+H₂)-containing gas product stream, includes two major steps, i.e., A) membrane separation followed by B) condensation and gas-liquid separation. Reversal of steps is also acceptable, that is, first condensation and gas-liquid separation, followed by membrane separation. The order of steps, either A→B or B→A, affects the management of process streams and recycle structure. But basically, whilst H₂S-rich streams generated by separation steps A→B or B→A are returned to the reforming reaction, H₂S-lean streams are jointly treated to further minimize H₂S level, then recover the products H₂ and CH₄. Thus, the invention provides a process comprising:
feeding sour natural gas mixed with H₂S-rich recycle streams, and optionally with CO₂, to an H₂S reforming reactor packed with a catalyst; catalytically reforming methane with H₂S and possibly CO₂ in said reactor;
   either passing the effluent from the reformer reactor through one or more membrane separator(s) to generate one or more permeate streams (rich with H₂S) and one or more retentate streams (lean with H₂S), recycling a permeate stream coming from a downstream membrane separator to the reformer reactor; condensing a retentate coming from an upstream membrane separator to recover liquid CS₂ and produce a non-condensable H₂S-rich stream, which is recycled to the reformer reactor, wherein during condensation, a non-condensable H₂S-lean stream is formed prior to the recovery of the liquid CS₂, and is optionally combined with a retentate stream coming from a downstream membrane separator; wherein the H₂S-lean stream or the combined H₂S-lean stream, is treated to recover H₂ and sweet natural gas therefrom;
   or vice versa, first condensing the effluent from the reformer reactor to recover liquid CS₂ and produce a non-condensable H₂S-rich stream, which is recycled to the reformer reactor, wherein, during condensation, a non-condensable H₂S-lean stream is formed prior to the recovery of the liquid CS₂; and passing the non-condensable H₂S-lean stream through one or more membrane separator(s) to generate one or more permeate streams and one or more retentate streams, recycling a permeate stream coming from a downstream membrane separator to the reformer reactor; and treating a retentate stream coming from a downstream membrane separator to recover H₂ and sweet natural gas therefrom.

H₂S-lean streams generated by the separation methods (A→B or B→A) are jointly treated to recover the products H₂ and CH₄; the treatment includes removal of residual acidic gases (H₂S, CO₂; for example, by absorption) to afford an essentially H₂S-free gas stream (e.g., not more than 4 ppm H₂S), recycling of the acidic gasses to the reforming reaction; optionally reduction of CO level by mixing the essentially H₂S-free gas stream with steam under conditions advancing water gas shift reaction; and ultimately, separation of H₂ and CH₄ from one another by membrane separation.

The description that follows focuses on the A→B order of steps.

One preferred variant of the invention, using an efficient recycle design of the H₂S and CO₂ streams, is a process for converting sour natural gas to sweet natural gas and producing hydrogen and carbon disulfide by H₂S reforming of methane to hydrogen and carbon disulfide, comprising:
feeding sour natural gas mixed with H₂S-rich recycle streams to an H₂S reforming reactor packed with a catalyst; catalytically reforming methane with H₂S in said reactor;
directing the reactor effluent into a two-stage membrane unit to separate H₂S-lean retentate from the first stage and H₂S-rich permeate from the second stage, recycling not less than 70-80% of the unreacted H₂S;
condensing the retentate coming from the first stage to form CS₂-containing condensed component and a first non-condensable component;
recycling said H₂S-rich permeate coming from the second stage to the reformer reactor; directing H₂S-lean retentate stream coming from the second stage and the first non-condensable component into an absorption unit to separate acidic gas and form an essentially H₂S-free product stream comprising methane, carbon monoxide, hydrogen and possibly carbon dioxide; recycling the acidic gas separated from the absorption unit to the reformer reactor;
recovering liquid CS₂ from the CS₂-containing condensed component, thereby producing a second non-condensable component, which contains H₂S and CH₄;
recycling the second non-condensable stream back to the reforming reactor;
feeding the sweet gas stream to a WGS reactor to convert CO and steam into CO₂ and hydrogen;
feeding the WGS reactor effluent to a membrane to separate hydrogen and CO₂ from the sweet natural gas; and
optionally combusting part of the sweet natural gas stream obtained, to supply heat to the reformer reactor; and
optionally recycling the CO₂ combustion product to the reformer reactor or supplying it to a plant where CO₂ and hydrogen are converted into liquid hydrocarbons, e.g., by the method described in U.S. Pat. No. 10,865,107 (sometimes named herein “the Blechner Center process”).

More specifically, the process of the invention comprises feeding sour natural gas and four recycle streams (possibly mixed with fresh CO₂ as needed) to a preheater then to a reformer packed with a solid catalyst. The projected H₂S conversion is at least 25%, e.g., from 35 to 50%, for example, about 40%. The effluent is cooled and compressed and fed to a two-stage membrane unit to separate a stream containing concentrated H₂S that is recycled to the reformer; the first retentate stream is cooled in a series of condensers to separate the by-product CS₂ in liquid form. One stream of non-condensable component of said retentate stream is fed together with the second retentate stream to an H₂S capture unit consisting of an absorption system or a membrane or a combination of the two; the condensable stream is fed to a separator at atmospheric pressure to separate liquid CS₂ and second non-condensable stream that is fed back to the reformer; the H₂S-rich stream from the H₂S capture unit is fed back to the reformer and the hydrogen-rich stream from the unit is fed into one, or two Water Gas Shift (WGS) reactors in series, together with steam to form CO₂ and hydrogen.

The WGS reaction takes place in one or more adiabatic fixed-bed reactors packed with a suitable WGS catalyst, for example, Cu/ZnO based low temperature water gas shift catalyst. The effluent is cooled by a condenser, and water is separated by a gas-liquid separator. The conditions in WGS reactor include WHSVco of not less than 1 h⁻¹, preferably not less than 2 h⁻¹. The reaction is carried out at a temperature in the range from 180 to 230 oC at a pressure of not less than 15 atmospheres, e.g., from 20 to 30.

The WGS reactor effluent stream is fed into a membrane that separates the sweet natural gas stream (containing <2% CO₂) from the hydrogen stream (containing CO₂ and methane); part of the sweet natural gas is combusted with oxygen to supply the heat for the reformer producing CO₂ that can optionally be recycled back to the reformer; the remaining sweet natural gas stream is fed into WGS reactor to reduce CO concentration below 4 ppm; the obtained hydrogen stream can be converted in the Blechner Center process (Herskowitz, Mordechay and Hos, Tomy, “Novel, highly efficient eco-friendly processes for converting CO₂ or CO-rich streams to liquid fuels and chemicals”, U.S. Pat. No. 10,865,107 (2020)) to produce green liquid fuels and chemicals. CO₂ generated in the process and separated from the very sour natural gas is reacted with hydrogen to produce green fuels and chemicals to minimize the carbon footprint of the process.

Turning now to FIG. 3, it is seen that the sour natural gas stream [1] is mixed with a recycle stream that combines stream [8] from the CS₂ separator (4), stream [10] from membrane (2), stream [13] from H₂S capture unit (5) and possibly CO₂ [28] from the combustion of natural gas (8). Fresh CO₂ [2] is mixed with the sour gas as needed to reach the desired CO₂/CH₄ ratio at the reformer inlet stream [3]. The combined stream [3] flows through a heat exchanger to raise the temperature, is fed to the reformer (1). The effluent stream [4] flows through the heat exchanger to cool down and transfer heat to stream [3]. The effluent stream [4] is further cooled down and pressurized by a compressor. H₂S is separated by one or two-stage membrane unit (2) and recycled back [10] to the reformer. CS₂ is condensed by cooling in a series of heat exchangers combined with gas-liquid separators (3). The incoming gas stream which enters (3) is at about room temperature; the gradual condensation is designed such that each heat exchanger accounts for 10-20 degrees reduction in the temperature of the gas, reaching −30° C. to −50° C. at the last gal-liquid separator (3), whereas the pressure of the gas is from 15 to 30 bar. The non-condensable stream [6] from system (3) containing mainly CH₄, H₂ and CO flows together with the second retentate stream [11], forming joint stream [12], to H₂S capture unit (5) that may consist of an amine-absorption tower or a membrane or the combination of the two to separate acid gas stream [13] that is recycled back to the reformer (1) and sweet gas stream [14]. The condensed stream from system (3) flows to gas-liquid separator (4) at atmospheric pressure. Liquid CS₂ is obtained in stream [7] and the non-condensable stream [8], rich in H₂S and CH₄, is recycled back to reformer (1). The sweet gas stream [14] from the H₂S capture unit (5) that contains mainly methane, hydrogen and carbon monoxide flows into two WGS reactors in series (6) together with steam [15] to convert CO into CO₂ and H₂, producing stream [16] that exits the second WGS reactor, passed through a heat exchanger and led to a gas-liquid separator. The effluent non-condensable stream [18] flows to a membrane (7) to separate a hydrogen-rich stream [19] and methane-rich, sweet natural gas stream [20]. Unreacted water [17] is removed.

Part of the sweet natural gas stream (26) is combusted (8) with oxygen [27] to supply the heat for the reformer and produce CO₂ that is recycled [28] to the reformer (1) or mixed [30] with stream [19]. The rich-hydrogen stream is used to produce higher hydrocarbons according to the process developed in the Blechner Center described in U.S. Pat. No. 10,865,107. The remaining sweet natural gas stream [21] flows to the WGS reactor, to react with steam [22], producing stream [23] that exits the WGS reactor, and led to a gas-liquid separator, to reduce CO concentration below 4 ppm in the treated natural gas stream [25]. Unreacted water [24] is removed.

By “catalyst activated in-situ by sulfidation” we mean a catalyst which undergoes activation in the reformer reactor; when 0.5-2.0 g of the catalyst are treated at a temperature in the range from 500 to 550° C., e.g., ˜530° C. and atmospheric pressure in a 50-150 mL min⁻¹ flow of 20 vol % H₂S/N₂ gas for 1 hour, the catalyst transforms into active phase(s) useful in catalyzing the conversion of sour natural gas. Examples of useful catalysts (in their form prior to activation) include:
One or more catalytically active metals, such as molybdenum, on a solid support, e.g., supported on alumina [Mo/γ-alumina]. For example, commercial γ-alumina (usually with BET surface area of 200-400 m²/g), is impregnated (optionally after calcination) with an aqueous or ethanolic solution of inorganic or organic molybdenum source, followed by drying and calcination at ˜450-550° C. For example, ammonium heptamolybdate tetrahydrate [(NH₄)₆Mo₇O₂₄·4H₂O] is dissolved in water in an alkaline environment (e.g., ammonium hydroxide) to form an impregnation solution for loading 2 to 20 wt. % of molybdenum, in its MoO₃ oxide form, onto the alumina support.
One or more catalytically active metals, such as molybdenum, on a solid support, alongside a promoter, e.g., one or more alkali metals, for example, potassium, all supported, e.g., on alumina [P—Mo-/γ-alumina, wherein P indicate the additional alkali metal acting as a promoter]. Mo is usually the major component of the combination. For example, the previously described Mo/γ-alumina is impregnated with an aqueous solution containing water soluble salt of K, e.g., K₂CO₃ solution, to load 2 to 15 wt. % of potassium onto the alumina support.
Metals (such as molybdenum) dispersed on the surface of a spinel compound. In its most general form, spinel is of the formula A²⁺(B³⁺)₂O₄ wherein A and B are divalent and trivalent metal cations, respectively. In a mixed spinel, at least two types of divalent metals (e.g., A₁²⁺, A₂²⁺, . . . ) or at least two types of trivalent metals (e.g., B₁³⁺, B₂³⁺, . . . ) are included in the spinel structure. That is, the mixed spinel that can be used in the invention is of the Formula 1:
(Aᵢ²⁺_{αi})·(Bⱼ³⁺_{βj})₂O₄, in which:
i is an integer equal to or greater than 1 (e.g., 1≤i≤4);
j is an integer equal to or greater than 1 (e.g., 1≤j≤4);

$$i + j \geq 3 ;$$

$$0 < ai \leq 1 , \sum ai = 1 ; and$$

$$0 < β j \leq 1 , \sum β j = 1.$$

The invention contemplates the use of catalysts composed of metals (such as

molybdenum) dispersed on the surface of spinel compounds of the formulas (A₁²⁺)·(B₁³⁺_{β1}B₂⁺_{β2})₂O₄, (A₁²⁺_{α1}A₂²⁺_{α2})·(B₁³⁺_{β1}B₂³⁺_{β2})₂O₄, (A₁²⁺_{α1}A₂²⁺_{α2}A₃²⁺_{α3})·(B₁³⁺_{β1}B₂³⁺_{β2})₂O₄, (A₁²⁺_{α1}A₂²⁺_{α2}A₃²⁺_{α3})·(B₁³⁺_{β1}B₂³⁺_{β2}B₃³⁺_{β3})₂O₄ and (A₁²⁺_{α1}A₂²⁺_{α2}A₃²⁺_{α3}A₄²⁺_{α4})·(B₁³⁺_{β1}B₂³⁺_{β2}B₃³⁺_{β3})₂O₄. For example, the divalent ion(s) Aᵢ²⁺ is selected from the group consisting of Ni²⁺, Co²⁺, Cu²⁺ and Zn²⁺ and the trivalent ion Bⱼ³⁺ is selected from the group consisting of Fe³⁺, Cr³⁺ and Al³⁺.

For example, Aᵢ²⁺ is Ni²⁺ and B₁³⁺ is Fe³⁺. The spinel compounds include: (Ni²⁺)·(Fe³⁺_{β1}B₂³⁺_{β2})₂O₄, in which B₂³⁺ is selected from Cr³⁺ and Al³⁺, with 0.1≤βj≤0.9; e.g., 0.25≤βj≤0.75;
(Ni²⁺_{α1}A₂²⁺_{α2})·(Fe³⁺_{β1}B₂³⁺_{β2})₂O₄, in which A₂²⁺ is selected from Co²⁺, Cu2⁺ and Zn²⁺, with 0.1≤αi≤0.9; e.g., 0.25≤αi≤0.75 and B₂³⁺ is selected from Cr³⁺ and Al³⁺ with 0.1≤βj≤0.9; e.g., 0.25≤βj≤0.75.

The spinel of Formula 1 exhibits surface area from, e.g., 10 to 50 m²/g, for example, from 10 to 20 m²/g, measured by the BET method.

Some of the spinel compounds are novel and form a separate aspect of the invention. X-ray diffraction analysis (using CuKα radiation) of samples of a few selected spinel compounds, e.g., Ni(Fe_{0.5}Cr_{0.5})₂O₄, (Ni_{0.5}Cu_{0.5})(Fe_{0.5}Cr_{0.5})₂O₄ (Ni_{0.5}Co_{0.5})(Fe_{0.5}Cr_{0.5})₂O₄ and (Ni_{0.5}Zn_{0.5}) (Fe_{0.5}Cr_{0.5})₂O₄ shows that the materials consist of a single spinel phase.

Table 1 lists the diffraction angles (2θ) and the [hkl] planes to which they refer.

TABLE 1

| | Ni(Fe_{0.5}Cr_{0.5})₂O₄ | (Ni_{0.5}Cu_{0.5})(Fe_{0.5}Cr_{0.5})₂O₄ | (Ni_{0.5}Co_{0.5})(Fe_{0.5}Cr_{0.5})₂O₄ | (Ni_{0.5}Zn_{0.5})(Fe_{0.5}Cr_{0.5})₂O₄ |
|---|---|---|---|---|
| hkl | Diffraction angles (2θ) | | | |
| 111 | 18.5 | 18.5 | 18.5 | 18.5 |
| 220 | 30.4 | 30.4 | 30.4 | 30.4 |
| 311 | 35.8 | 35.8 | 35.7 | 35.8 |
| 222 | 37.4 | 37.4 | 37.4 | 37.5 |
| 400 | 43.5 | 43.5 | 43.4 | 43.6 |
| 422 | 54.0 | 54.0 | 53.9 | 54.1 |
| 511 | 57.6 | 57.6 | 57.5 | 57.7 |
| 440 | 63.2 | 63.2 | 63.1 | 63.3 |
| 531 | 66.5 | 66.5 | 66.4 | 66.7 |

The spinel of Formula 1 exhibits catalytic activity by itself. However, it was found that the performance of Ni(Co,Zn,Cu)—Fe(Cr,Al)—O spinel catalyst in direct catalytic conversion of sour natural gas is significantly improved after deposition on its surface nanocrystals of MoO3 oxide phase thus forming a catalyst by Formula 2:
xMoO₃/(Aᵢ²⁺_{αi})·(Bⱼ³⁺_{βj})₂O₄, wherein the x is in range of 1.0 to 50.0 wt. %, e.g. from 1-20 wt. %. For example:

xMoO₃/(A₁²⁺)·(B₁³⁺_{β1}B₂³⁺_{β2})₂O₄;

xMoO₃/(A₁²⁺_{α1}A₂²⁺_{α2})·(B₁³⁺_{β1}B₂³⁺_{β2})₂O₄;
xMoO₃/(A₁²⁺_{α1}A₂²⁺_{α2}A₃²⁺_{α3})·(B₁³⁺_{β1}B₂³⁺₂)₂O₄;

xMoO₃/(A₁²⁺_{α1}A₂²⁺_{α2}A₃²⁺_{α3})·(B₁³⁺_{β1}B₂³⁺_{β2}B₃³⁺_{β3})₂O₄; and

xMoO₃/(A₁²⁺_{α1}A₂²⁺_{α2}A₃²⁺_{α3}A₄²⁺_{α4})·(B₁³⁺_{β1}B₂³⁺_{β2}B₃³⁺_{β3})₂O₄.

The spinel of Formula 1 is prepared by the sol-gel method, assisted by a complexing agent. For example, the method of synthesis of the compound (A₁²⁺)19 (B₁³⁺_{β11}B₂³⁺_{β2})₂O₄, such as Ni²⁺(Fe³⁺_{β1}Cr³⁺_{β2})₂O₄, comprises dissolution in water of the A₁²⁺, B₁³⁺ and B₂³⁺ (e.g., Ni²⁺, Fe³⁺ and Cr³⁺) salt precursors, e.g., nitrate salts, to form Ni²⁺, Fe³⁺and Cr³⁺solution (this could also be done by combining separate aqueous solutions of the individual salts), adding a complexing agent, such as citric acid, to the mixed salt solution, heating the mixture to 60-90° C. until the gel is formed. The spinel powder is recovered by drying the material overnight at ˜110° C., followed by two calcination cycles, first in air at ˜200° C. (e.g., at 10° C./min) for 1-3 h and then at 500-700° C. (5° C./min) for 4 h.

The catalyst of Formula 2 is prepared by loading the catalytically active metal component, e.g., molybdenum, onto the spinel surface by impregnation. That is, the spinel powder is impregnated with an aqueous solution of Mo precursor salt (suitable Mo sources were described above), followed by drying (e., first in air then in the oven) and calcination (e.g., >500° C.). Illustrative conditions are provided in the experimental section below.

Another aspect of the invention is an apparatus suitable for converting sour natural gas to sweet natural gas, hydrogen and carbon disulfide, comprising:
a reformer reactor (1), packed with a catalyst, supplied by a feed line [1] from a sour natural gas reservoir, optionally by a feed line [2] connected to an external fresh CO2 source; and by one or more recycle lines (i.e., at least one of [8], [10] and [13];
a separation unit (S) connected to the outlet of the reformer reactor (1) through a line [4] equipped with a heat exchanger and a compressor; the separation unit consisting of membrane separator(s) (2), gas-liquid separators arranged in series, with a heat exchanger in the line connecting a pair of adjacent gas-liquid separators (3) and a terminus gas-liquid separator (4);
acidic gas removal unit (5), which comprises either an absorption unit filled with a liquid, suitable for separating a gas mixture passing therethrough by dissolving one or more acidic components of the mixture, or a membrane separator; wherein the acidic gas removal unit (5) is supplied by one or more feed lines from the separation unit(S), and is connected by a recycle line [13] to the reformer reactor (1) and through a product delivery line [14] to a WGS reactor (6);
One or more WGS reactors in series (6), wherein the first WGS reactor is supplied with a steam feed line [15] and a feed line [14] that is connected to the outlet of the acidic gas removal unit (5), to deliver one or more gas components which were not captured in the acidic gas removal unit, to said first WGS reactor;
hydrogen separation membrane unit (7), configured to receive a non-condensable component of the effluent of the first WGS reactor, or the last WGS reactor in said series of WGS reactors, wherein the permeate side of said hydrogen separation membrane unit (7) is connected [19] to a plant suitable for producing liquid hydrocarbons from hydrogen and CO₂; such that hydrogen and CO₂-containing permeate generated in said membrane can be used as a feed material in production of liquid hydrocarbons in said plant;
a combustion chamber (8), connected [20, 26] to the retentate side of said hydrogen separation membrane unit (7), to receive CH₄-containing stream, wherein the combustion chamber is supplied by an oxygen feed line [27], wherein the combustion chamber is linked to the reformer reactor to supply heat by radiation and convection, and deliver CO₂ combustion product [28] to the inlet of said reactor or as a feed material [30] in production of liquid hydrocarbons;
an WGS reactor connected [20, 21] to the retentate side of said hydrogen separation membrane unit (7), to receive CH₄ and CO-containing stream, wherein the WGS reactor is supplied with a steam feed line [22], with gas-liquid separator placed downstream of said WGS reactor to recover sweet natural gas [25] and water [24];
   wherein the separation unit (S) consists of:
A) a single or multistage membrane separator(s) (2);
B) n gas-liquid separators positioned in series (3₁, . . . , 3ₙ; n≥2; e.g., 3≤n≤7), wherein lines delivering condensates from said gas-liquid separators (3₁, . . . , 3ₙ) are joined to provide a feed line for the terminus gas-liquid separator (4) which is configured to operate under atmospheric pressure, wherein the liquid discharge line [7] of said terminus gas-liquid separator (4) is connected to a storage tank for holding CS₂, with a recycle line [8] connecting the gas outlet of said terminus gas-liquid separator (4) to the reformer reactor (1); wherein
   either A is upstream of B, in which case:
the condenser (3ₙ) is connected [6] by a pipe to supply non-condensable matter to acidic gas removal unit (5); and
when A) consists of a single membrane separator (2), then the retentate side of said single membrane separator is connected to the inlet of the first gas-liquid separator (3₁), and the permeate side of said single membrane separator (2) is connected to the reformer reactor; or
when A) consists of a multistage membrane separator (2), then the retentate and permeate sides of the first stage membrane separator are connected to the inlet of the first gas-liquid separator (31) and to a second stage membrane separator, respectively, and for any stage other than the first stage, the permeate side is connected by recycle line [10] to the reformer reactor (1) and the retentate side is either connected to the next stage or, in case of the last stage, to an acidic gas removal unit (5) via pipe [11];
   or A is downstream to B, in which case then the gas-liquid contactor (3ₙ) is connected by a pipe to supply non-condensable matter to a single or multistage membrane separator(s) (2).

### BRIEF DESCRIPTION OF THE DRAWINGS

FIG. 1 displays block diagram for sour natural gas processing adopted from Mansi S. Shah, Michael Tsapatsis and J. Ilja Siepmann, “Hydrogen Sulfide Capture: From Absorption in Polar Liquids to Oxide, Zeolite, and Metal-Organic Framework Adsorbents and Membranes”, Chem. Rev. 2017, 117, 9755-9803.

FIG. 2 displays the thermodynamic analysis of hydrogen sulfide reforming with methane using CHEMCAD software at 1 bar from 700 to 1000° C. with inlet feed ratio of H₂S/CH₄0.6 and CO₂/CH₄=0.3 with products CS₂, H₂ and CO.

FIG. 3 displays the design of the process of the invention.

FIG. 4 depicts a schematic description of an experimental set up for H₂S reforming of methane.

FIG. 5 displays the XRD patterns of the CrNiFeO₄ catalyst sample.

FIG. 6 displays the XRD patterns of the CrNiCuFeO₄ catalyst sample.

FIG. 7 displays the XRD patterns of the CrNiCoFeO₄ catalyst sample.

FIG. 8 displays the XRD patterns of the CrNiZnFeO₄ catalyst sample.

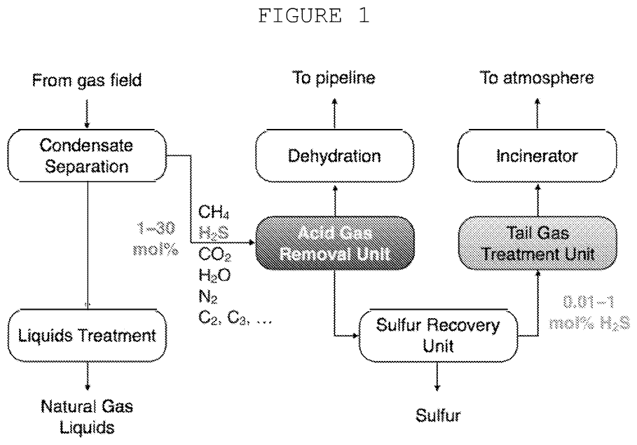

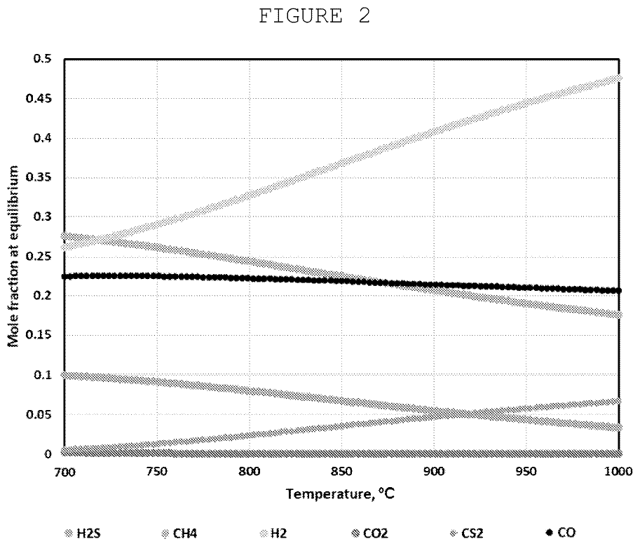

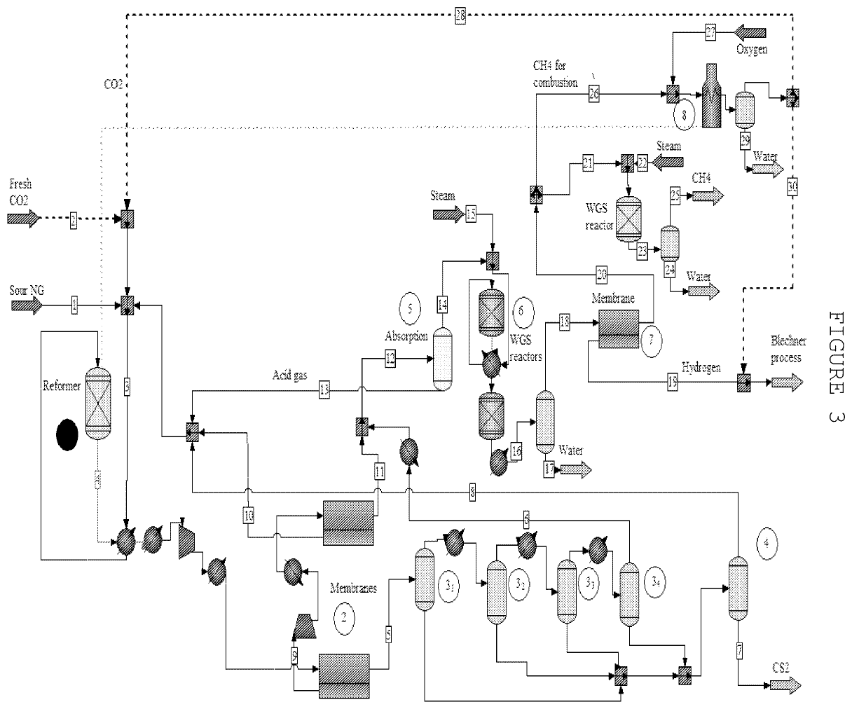

### DETAILED DESCRIPTION

Conventional wide-angle XRD patterns were measured with a Panalytical Empyrean Powder Diffractometer equipped with position sensitive detector X'Celerator fitted with a graphite monochromator, at 40 kV and 30 mA and analyzed using software developed by Crystal Logic. The phase identification was performed by using an SBDE ZDS computer search/match program coupled with the International Center for Diffraction Data (ICDD). BET was measured by NOVA 3200e Quantachrome adsorption analyzer.

### Preparation 1

### Mo/γ-Al₂O₃ (20 wt. % MoO₃)

Mo/γ-Al₂O₃ catalyst was prepared by incipient wetness impregnation. 8 g of γ-Al₂O₃ support (NORTON, SA6175, ⅛″ extrudates, 230-290 m²/g) were calcined at 500° C. for 2 h prior to impregnation. After calcination the support was held under vacuum for 1 h and further impregnated with solution of 2.454 g (NH₄)₆Mo₇O₂₄·4H₂O in 4.25 g H₂O and 1.5 ml NH₄OH 25%. The material was dried at room temperature for 24 h. Next, the catalyst was dried at 120° C. for 12 h and calcined at 550° C. for 4 h (3° C./min).

### Preparation 2

### K-Mo/γ-Al₂O₃ (20 wt. % MoO₃, 5 wt. % K)

K-Mo/γ-Al₂O₃ catalyst was prepared by incipient wetness impregnation in two steps. At first step Mo/γ-Al₂O₃ material was prepared according to previous synthesis (Preparation 1). After that, the 10.67 g of obtained material was held under vacuum for 1 h and further impregnated with solution of 0.988 g K₂CO₃ in 5.85 g H2O. The catalyst was dried at 120° C. overnight and calcined at 500° C. for 2 h (5° C./min). BET surface area was 166 m²/g.

### Preparation 3

### Mo/Ni(Fe_{0.5}Cr_{0.5})₂O₄ (5 wt. % MoO₃)

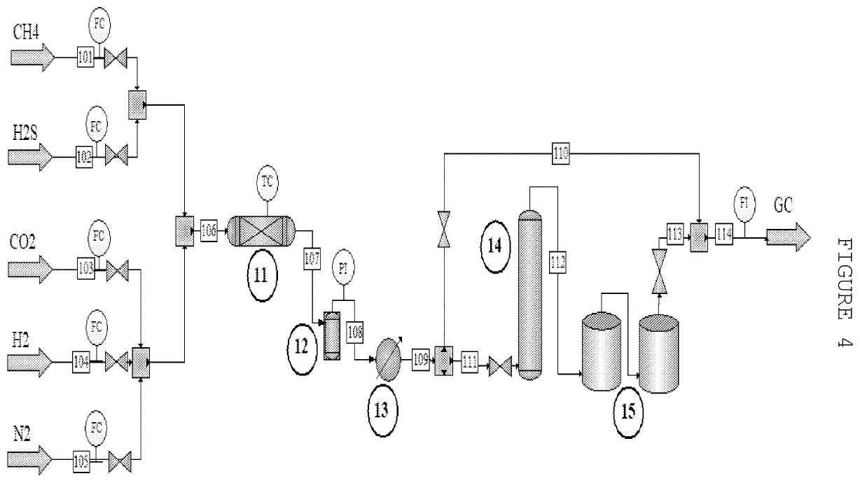

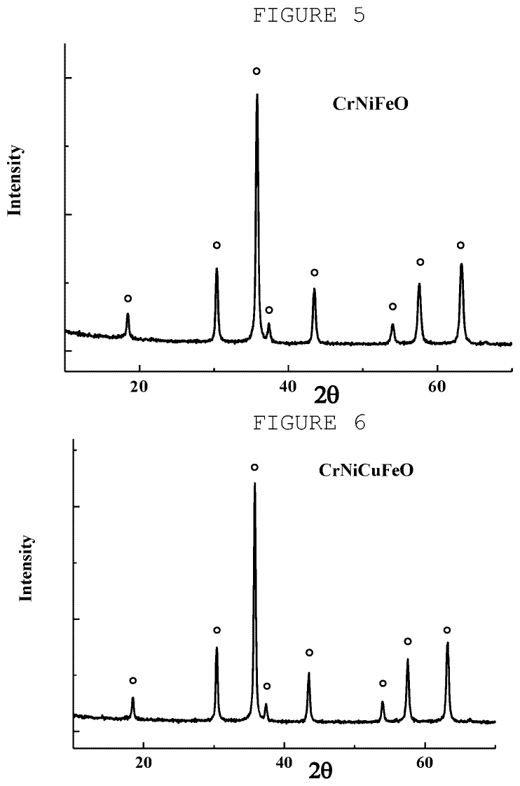

Ni(Fe_{0.5}Cr_{0.5})₂O₄ material was synthesized by the sol-gel method. The metal salt precursors were dissolved separately in 10 ml H₂O each: 8.591 g Cr (NO₃)₃·9H₂0; 6.570 g Ni(NO₃)₂·6H₂O; 9.128 g Fe(NO₃)₃·9H₂O. Once fully dissolved, the metal precursors were mixed and 32.566 g of citric acid (complexant) was added to the solution (the ratio of moles complexant to total moles of metal ions was 2.5). Then the mixed solution was heated to 80° C. on a hot plate until the gel was formed. The material was dried overnight at 110° C., calcined in air at 200° C. (10° C./min) for 2 h and then at 700° C. (5° C./min) for 4 h. X-ray powder diffraction is shown in FIG. 5. BET surface area was 17 m²/g.

4.61 g of Ni(Fe_{0.5}Cr_{0.5})₂O₄ material was held under vacuum for 1 h and impregnated with solution of 0.298 g (NH₄)₆Mo₇O₂₄·4H₂O in 3.24 g H₂O and 0.18 ml NH₄OH 25%. The material was dried at room temperature for 24 h. After that the catalyst was dried at 120° C. for 12 h and calcined at 550° C. for 4 h (3° C./min).

### Preparation 4

### Mo/(Ni_{0.5}Cu_{0.5})(Fe_{0.5}C_{0.5})₂O₄ (5 wt. % MoO₃)

(Ni_{0.5}Cu_{0.5})(Fe_{0.5}Cr_{0.5})₂O₄ material was synthesized by the sol-gel method. The metal salt precursors were dissolved separately in 10 ml H₂O each: 8.591 g Cr(NO₃)₃·90_{H}2O; 3.286 g Ni(NO₃)₂·6H₂O; 9.128 g Fe(NO₃)₃·9H₂O; 2.730 g Cu(NO₃)₂·3H₂O. Once fully dissolved, the metal precursors were mixed and 32.566 g of citric acid (complexant) was added to the solution (the ratio of moles complexant to total moles of metal ions was 2.5). Then the mixed solution was heated to 80° C. on a hot plate until the gel was formed. The material was dried overnight at 110° C., calcined in air at 200° C. (10° C./min) for 2 h and then at 700° C. (5° C./min) for 4 h. X-ray powder diffraction is shown in FIG. 6.

4.66 g of (Ni_{0.5}Cu_{0.5})(Fe_{0.5}Cr_{0.5})₂O₄ material was held under vacuum for 1 h and impregnated with solution of 0.286 g (NH₄)₆Mo₇O₂₄·4H₂O in 2.88 g H₂O and 0.18 ml NH₄OH 25%. The material was dried at room temperature for 24 h. After that the catalyst was dried at 120° C. for 12 h and calcined at 550° C. for 4 h (3° C./min).

### Preparation 5

### Mo/(Ni_{0.5}Co_{0.5})(Fe_{0.5}Cr_{0.5})₂O₄ (5 wt. % MoO₃)

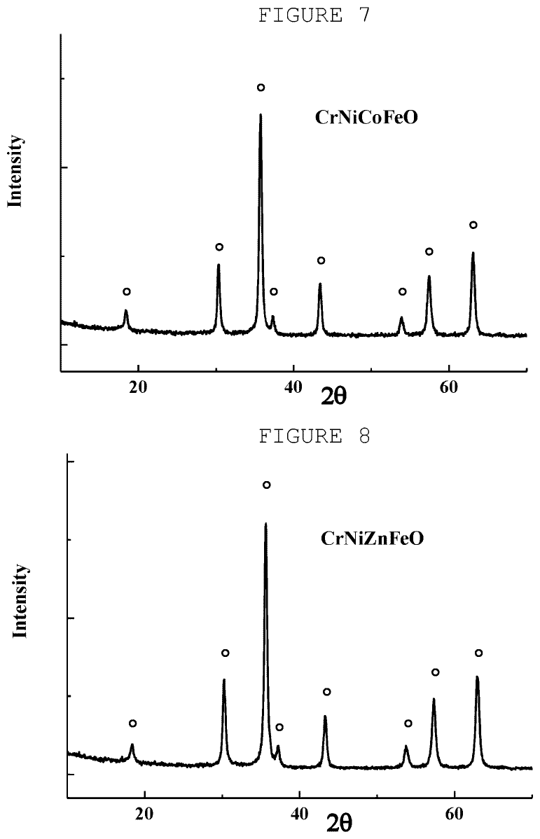

(Ni_{0.5}Co_{0.5})(Fe_{0.5}Cr_{0.5})₂O₄ material was synthesized by the sol-gel method. The metal salt precursors were dissolved separately in 10 ml H₂O each: 8.591 g Cr(NO₃)₃·9H₂O; 3.286 g Ni(NO₃)₂·6H₂O; 9.128 g Fe(NO₃)₃·9H₂O; 3.288 g Co(NO₃)₂·6H₂O. Once fully dissolved, the metal precursors were mixed and 32.566 g of citric acid (complexant) was added to the solution (the ratio of moles complexant to total moles of metal ions was 2.5). Then the mixed solution was heated to 80° C. on a hot plate until the gel was formed. The material was dried overnight at 110° C., calcined in air at 200° C. (10° C./min) for 2 h and then at 700° C. (5° C./min) for 4 h. X-ray powder diffraction is shown in FIG. 7.

3.58 g of (Ni_{0.5}Co_{0.5})(Fe_{0.5}Cr_{0.5})₂O₄ material was held under vacuum for 1 h and impregnated with solution of 0.219 g (NH₄)₆Mo₇O₂₄·4H₂O in 1.58 g H20 and 0.11 ml NH4OH 25%. The material was dried at room temperature for 24 h. After that the catalyst was dried at 120° C. for 12 h and calcined at 550° C. for 4 h (3° C./min).

### Preparation 6

### Mo/(Ni_{0.5}Zn_{0.5})(Fe_{0.5}Cr_{0.5})₂O₄ (5 wt. % MoO₃)

(Ni_{0.5}Zn_{0.5})(Fe_{0.5}Cr_{0.5})₂O₄ material was synthesized by the sol-gel method. The metal salt precursors were dissolved separately in 10 ml H2O each: 8.591 g Cr(NO₃)₃·9H₂O; 3.286 g Ni(NO₃)₂·6H₂O; 9.128 g Fe(NO₃)₃·9H₂O; 2.480 g Zn(CH₃COO)₂·2H₂O. Once fully dissolved, the metal precursors were mixed and 32.566 g of citric acid (complexant) was added to the solution (the ratio of moles complexant to total moles of metal ions was 2.5). Then the mixed solution was heated to 80° C. on a hot plate until the gel was formed. The material was dried overnight at 110° C., calcined in air at 200° C. (10° C./min) for 2 h and then at 700° C. (5° C./min) for 4 h. X-ray powder diffraction is shown in FIG. 8.

4.79 g of (Ni_{0.5}Zn_{0.5})(Fe_{0.5}Cr_{0.5})₂O₄ material was held under vacuum for 1 h and impregnated with solution of 0.293 g (NH₄)₆Mo₇O₂₄·4H₂O in 4.23 g H₂O and 0.23 ml NH₄OH 25%. The material was dried at room temperature for 24 h. After that the catalyst was dried at 120° C. for 12 h and calcined at 550° C. for 4 h (3° C./min).

### Examples 1 and 2

### H₂S Reforming of Methane with Complete Conversion of CO₂ in a Fixed Bed Reactor

A schematic description of the experimental set-up used for running the H₂S reforming of methane is depicted in FIG. 4. Catalyst activation was done by in-situ sulfidation at temperature of 530° C. and atmospheric pressure in reactor (11) at 200 mL min⁻¹ flow of 20 vol % H₂S/N₂ gas for 1 hour. The gaseous reactants are controlled by Brooks mass flow controllers (FC).

Methane was contacted with H₂S and CO₂ by passing a mixture of CH₄, H₂S and CO₂ streams (indicated by numerals [101], [102] and [103], respectively) at a molar ratio H₂S/CH₄ and CO₂/CH₄ of 0.6 and 0.3, respectively, through a tubular reactor (11) (11 mm ID, 600 mm long) made of alumina, packed with 1.5 gram of the catalyst powder of Preparation 1 (Mo/γ-Al₂O₃) or 0.75 gram of the catalyst powder of Preparation 2 (K-Mo/γ-Al₂O₃) and 4 gram of quartz powder and heated up to 900° C. at a total pressure of 1 atm (Examples 1 and 2, respectively). All gaseous reactants are fed via line [106] to the reactor (11).

The reaction products are cooled down to 150° C. with the aid of an electric heater (12) to separate and capture sulfur residues, which may form in the H₂S decomposition reaction. With the aid of a cooler (13), the gaseous products [108] were cooled down to 5° C. The gaseous products [109] flow in line [110] to GC analyzer or to an absorption column (14) containing paraffinic solvent to absorb most of the CS₂ produced in the reformer (11). The effluent gas stream [112] containing H₂S, CS₂, CH₄, CO₂, CO and H₂ is fed into two scrubber vessels in series (15) containing 2 L of sodium hydroxide solution, to remove H₂S, CO₂ and CS₂ effectively.

The exhaust components [113] flowing in line [114] were analyzed in online Agilent 7890A Series Gas Chromatograph (GC) equipped with 7 columns and 5 automatic valves using helium as a carrier gas. The flow rate was measured by Alicat mass flow meter (FI).

In the tables below, the capital letters X and S stand for conversion and selectivity, respectively. The selectivity towards H₂S reforming reaction was calculated as S_{H2S_reforming}=[(X_{H2S(reforming)}*λ/2)/X_{CH4}], where λ is the H₂S/CH₄ molar ratio at feed.

The reaction of H₂S (H₂S reforming) and CO₂ (dry reforming) with CH₄ in reactor (11) to produce H₂, CO and CS₂ was run under specific conditions at close to equilibrium conversions: Temperature of 900° C., total pressure of 1 atm, H₂S/CH₄=0.6 mol/mol and CO₂/CH₄=0.3 mol/mol. Results are shown in Table 2.

TABLE 2

| | | Time on | WHSV_{H2S}, | X_{CH4}, | X_{H2S}; | X_{CO2}, | |
|---|---|---|---|---|---|---|---|
| Example | Catalyst | stream, h | h⁻¹ | % | % | % | S_{H2S}_{_{—}}_{reforming} |
| 1 | Mo/γ-Al₂O₃ | 50 | 2.0 | 43.2 | 45.8 | 100.0 | 31.8 |
| 2 | K-Mo/γ-Al₂O₃ | 100 | 4.0 | 43.5 | 46.7 | 100.0 | 32.2 |

### Examples 3-5

### H₂S Reforming of Methane with Low Conversion of CO₂ in a Fixed Bed Reactor

This experiment was conducted in an experimental unit with a similar design as in Examples 1-2, schematically described in FIG. 4. The tubular reactor (11) (11 mm ID, 600 mm long) was packed with 2 grams of the catalyst powder of Preparation 3 (Mo/Ni (Fe_{0.5}Cr_{0.5})₂O₄) or 1.5 gram of Preparation 6 (Mo/(Ni_{0.05}Zn_{0.5})(Fe_{0.5}Cr_{0.5})₂O₄) and 5 gram of quartz powder.

CO₂ [103] and H₂S [102] streams were contacted with CH₄ [101] to form a feed mixture [106] containing H₂S/CH4 and CO₂/CH₄ in a molar ratio of 0.4-0.6 and 0.08-0.30, respectively. CO₂ was partially reacting with CH₄ and H₂ in the dry reforming and RWGS reactions, respectively.

The reaction was run under the following specific conditions:
Temperature 900° C. and total pressure of 1 atm. Conditions and results are shown in Table 3.

TABLE 3

| | | Time on | | | | | | | |
|---|---|---|---|---|---|---|---|---|---|
| | | Stream | CO₂/CH₄ | H₂S/CH₄ | WHSV_{H2} | X_{CH4} | X_{H2S} | X_{CO2} | |
| Ex. | Catalyst | (h) | mol/mol | mol/mol | h⁻¹ | % | % | % | S_{H2S}_{_{—}}_{reforming} |
| 3 | Mo/Ni(Fe_{0.5}Cr_{0.5})₂O₄ | 45 | 0.30 | 0.6 | 0.6 | 13.5 | 30.0 | 11.0 | 66.7 |
| 4 | Mo/Ni(Fe_{0.5}Cr_{0.5})₂O₄ | 165 | 0.15 | 0.6 | 1.3 | 9.8 | 29.2 | 3.0 | 89.7 |
| 5 | Mo(Ni_{0.5}Zn_{0.5})(Fe_{0.5}Cr_{0.5})₂O₄ | 45 | 0.08 | 0.4 | 0.9 | 8.0 | 25.1 | 25.0 | 62.8 |

### Example 6

### H₂S Reforming of Methane Mixed with CO₂ and H₂ in a Fixed Bed Reactor

This experiment was conducted in an experimental unit with a similar design as in Examples 1-2 schematically described in FIG. 4. The tubular reactor (11) (11 mm ID, 600 mm long) was packed with 1.5 grams of the catalyst powder of Preparation 1 (Mo/γ-Al₂O₃) and 4 gram of quartz powder.

H₂S [102], CO₂ [103] and H₂ [104] streams were contacted with CH₄ [101] to evaluate the performance of the projected gas composition with hydrogen at feed mixture [106].

The reaction was run under the following specific conditions:
WHSV_{H2S}=2.0 h⁻¹, temperature 900° C., total pressure of 1 atm, 8% v/v H₂, H₂S/CH₄=0.6 mol/mol and CO₂/CH₄=0.3 mol/mol in the feed. The time on stream was 70 hours. The results are shown in Table 4.

TABLE 4

| X_{CH4}, % | X_{H2S}, % | x_{CO2}, % | S_{H2S}_{_{—}}_{reforming}, % |
|---|---|---|---|
| 38.0 | 40.0 | 86.6 | 31.6 |

### Example 7

### H₂S Reforming of Methane Without CO₂ at Feed in a Fixed Bed Reactor

This experiment was conducted in an experimental unit with a similar design as in Examples 1-2 schematically described in FIG. 4. The tubular reactor (11) (11 mm ID, 600 mm long) was packed with 2 grams of the catalyst powder of Preparation 3 (Mo/Ni (Fe_{0.5}Cr_{0.5})₂O₄) and 5 gram of quartz powder.

H₂S [102] stream was contacted with CH₄ [101] to form a feed mixture [106] containing H₂S/CH₄ in a molar ratio of 0.6, without CO₂ in feed.

The reaction was run under the following specific conditions:
WHSV_{H2S}=0.6 h⁻¹, temperature 900° C., total pressure of 1 atm, H₂S/CH₄=0.6 mol/mol in the feed. The time on stream was 75 hours. The results are shown in Table 5.

TABLE 5

| X_{CH4}, % | X_{H2S},% |
|---|---|
| 12.8 | 39.5 |

While the present disclosure has been illustrated and described with respect to a particular embodiment thereof, it should be appreciated by those of ordinary skill in the art that various modifications to this disclosure may be made without departing from the spirit and scope of the present disclosure.

1. A process for preparing hydrogen by a catalytic conversion of sour natural gas, comprising feeding sour natural gas and one or more H₂S recycled streams, optionally mixed with fresh CO₂, to a reformer reactor packed with a catalyst activated in-situ by sulfidation.

2. The process according to claim 1, wherein the feed stream comprises from 50 to 90 vol % methane, not less than 10 vol % H₂S and 0 to 40 vol % CO₂.

3. The process according to claim 2, wherein the sour natural gas comprises from 15 to 35 vol % H₂S.

4. The process according to claim 1, wherein the catalytic conversion of sour natural gas takes place over the catalyst in the reformer reactor under the following conditions:
temperature in the range from 800 to 950° C., WHSV_{H2S} in the range of 0.5 to 5 h⁻¹, at total pressure of 1 to 3 atm.

5. The process according to claim 1, wherein the effluent from the reactor is passed through a separation system comprising several units; H₂S-containing streams are collected and are recycled to the reformer reactor, whereas CO₂ is produced downstream and is directed to the reformer reactor or is used as a feed component, together with the hydrogen produced by the process, in a plant converting hydrogen and CO₂ into liquid hydrocarbons.

6. The process according to claim 1, wherein separation of unreacted H₂S from the effluent of the reformer reactor, and separation of the effluent into a liquid stream consisting of the Cₛ2 by-product and the (CH₄+H₂)-containing gas product stream, includes:
A) membrane separation followed by B) condensation and gas-liquid separation; or
B) condensation and gas/liquid separation followed by A) membrane separation;
wherein H₂S-rich streams generated by separation steps A→B or B→A are returned to the reforming reactor and H₂S-lean streams are jointly treated to further minimize H₂S level, then recover the products H₂ and CH₄ therefrom.

7. The process according to claim 6, comprising:
feeding sour natural gas mixed with H₂S-rich recycle streams, and optionally with CO₂, to a H₂S reforming reactor packed with a catalyst; catalytically reforming methane with H₂S in said reactor;
either passing the effluent from the reformer reactor through one or more membrane separator(s) to generate one or more permeate streams (rich with H₂S) and one or more retentate streams (lean with H₂S), recycling a permeate stream coming from a downstream membrane separator to the reformer reactor; condensing a retentate coming an upstream membrane separator to recover liquid CS₂ and produce a non-condensable H₂S-rich stream, which is recycled to the reformer reactor, wherein during condensation, a non-condensable H₂S-lean stream is formed prior to the recovery of the liquid CS₂, and is optionally combined with a retentate stream coming from an downstream membrane separator; wherein the H₂S-lean stream, or the combined H₂S-lean stream, is treated to recover H₂ and sweet natural gas therefrom;
or vice versa, first condensing the effluent from the reformer reactor to recover liquid CS₂ and produce a non-condensable H₂S-rich stream, which is recycled to the reformer reactor, wherein, during condensation, a non-condensable H₂S-lean stream is formed prior to the recovery of the liquid CS₂; and passing the non-condensable H₂S-lean stream through one or more membrane separator(s) to generate one or more permeate streams and one or more retentate streams, recycling a permeate stream coming from a downstream membrane separator to the reformer reactor; and treating a retentate stream coming from a downstream membrane separator to recover H₂ and sweet natural gas therefrom.

8. The process according to claim 7, wherein H₂S-lean streams generated by the separation methods (A→B or B→A) are jointly treated to recover the products H₂ and CH₄ by removal of residual acidic gases to afford an essentially H₂S-free gas stream, recycling of the acidic gasses to the reformer reactor; optionally reducing CO level by mixing the essentially H₂S-free gas stream with steam under conditions advancing water gas shift reaction; and ultimately, separating H₂ and CH₄ from one another by membrane separation.

9. The process according to claim 1, for converting sour natural gas to sweet natural gas and producing hydrogen and carbon disulfide by H₂S reforming of methane to hydrogen and carbon disulfide, comprising:
feeding sour natural gas [1] mixed with H₂S-rich recycle streams [8], [10], [13] and optionally with CO₂ [28], to a H₂S reforming reactor (1) packed with a catalyst; catalytically reforming methane with H₂S in said reactor;
directing the reactor effluent [4] into a two-stage membrane unit (2) to separate H₂S-lean retentate [5] from the first stage and H₂S-rich permeate [10] from the second stage;
condensing the retentate [5] coming from the first stage to form Cₛ2-containing condensed component and a first non-condensable component;
recycling said H₂S-rich permeate [10] coming from the second stage to the reformer reactor (1);
directing H₂S-lean retentate stream [11] coming from the second stage and the first non-condensable component [6] into an absorption unit (5) to separate acidic gas stream [13] and form an essentially H₂S-free, sweet gas product stream [14] comprising methane, carbon monoxide, hydrogen and possibly carbon dioxide;
recycling the acidic gas [13] separated from the absorption unit to the reformer reactor (1);
recovering liquid CS₂ [7] from the CS₂-containing condensed component, thereby producing a second non-condensable component [8], which contains H₂S and CH₄;
recycling the second non-condensable stream [8] back to the reforming reactor (1);
feeding the sweet gas stream [14] to a WGS reactor (6) to convert CO and steam [15] into CO₂ and hydrogen;
feeding the WGS reactor gas effluent [18] to a membrane to separate hydrogen and CO₂ [19] from the sweet natural gas; and
optionally combusting part of the sweet natural gas stream [26] obtained, to supply heat to the reformer reactor; and
optionally recycling the CO₂ combustion product to the reformer reactor [28] or supplying it [30] to a plant where CO₂ and hydrogen are converted into liquid hydrocarbons.

10. The process according to claim 1, wherein the catalyst is selected from the group consisting of:
i) one or more catalytically active metals on a solid support;
ii) one or more catalytically active metals on a solid support, alongside a promoter.
iii) a spinel compound, optionally with one or more catalytically active metals dispersed on the surface of the spinel compound, which has the formula (1)
(Aᵢ²⁺_{αi})·(Bⱼ³⁺_{βj})₂O₄ Formula (1)
wherein:
Aᵢ²⁺ is a bivalent metal; Bⱼ³⁺ is a trivalent metal; 1≤i≤4; 1≤j≤4; i+j>3; 0<αi≤1, Σ αi=1; 0<βj≤1, Σ βBj=1.

11. The process according to claim 10, wherein the catalyst is selected from the group consisting of:
i) molybdenum on a solid support;
ii) molybdenum on a solid support, alongside a promoter, which is potassium;
iii) a spinel compound of Formula 1 selected from the group consisting of: (A₁²⁺)·(B₁³⁺_{β1}B₂³⁺_{β2})₂O₄, (A₁²⁺_{α1}A₂⁺_{α2}d )·(B₁³⁺_{β1}B₂³⁺_{β2})₂O₄, (A₁²⁺_{α1}A₂²⁺_{α2}A₃²⁺_{α3})—(B₁³⁺_{β1}B₂³_{β2})₂O₄, (A₁²⁺_{α1}A₂²⁺_{α2}A₃²⁺_{α3})·(B₁³⁺_{β1}B₂³⁺_{β2}B₃³⁺_{β3})₂O₄ and (A₁²⁺_{α1}A₂²⁺_{α2}A₃²⁺_{α3}A₄²⁺_{α4})·(B₁³⁺_{β1}B₂³⁺_{β2}B₃³⁺_{β3})₂O₄,
wherein the divalent metal A₁²⁺ is selected from the group consisting Ni²⁺, Co²⁺, Cu²⁺ and Zn²⁺, the trivalent metal Bⱼ³⁺ is selected from the group consisting of Fe³⁺, Cr³⁺ and Al³⁺; 0<αi≤1, Σ αi=1, 0<βj≤1, Σ j=1, wherein molybdenum is dispersed on the spinel.

12. The process according to claim 11, wherein the catalyst is selected from the group consisting of:
i) Mo/γ-alumina;
ii) K-Mo-/γ-alumina]; and
iii) Mo/Ni(Fe_{0.5}Cr_{0.5})₂O₄, Mo/(Ni_{0.5}Cu_{0.5})(Fe_{0.5}Cr_{0.5})₂O₄, Mo/(Ni_{0.5}Co_{0.5})(Fe_{0.5}Cr_{0.5})₂O₄ and Mo/(Ni_{0.5}Zn_{0.5})(Fe_{0.5}Cr_{0.5})₂O₄.

13. A spinel compound of Formula 1:
(A₁²⁺_{α1})·(Bⱼ³⁺_{β2})₂O₄ Formula (1)
selected from the group consisting of:
(A₁²⁺)·*B₁³⁺_{β1}B₂^{3°}_{β2})₂O₄;
(A₁²⁺_{α1}A₂²⁺_{α2})·(B₁³⁺_{β1}B₂³⁺_{β2})₂O₄;
(A₁²⁺_{α1}A₂²⁺_{α2}A₃²⁺_{α3})·(B₁³⁺_{β1}B₂^{3 +}_{β2})₂O₄;
(A₁²⁺_{α1}A₂²⁺_{α2}A₃²⁺_{α3})·(B₁³⁺_{β1}B₂³⁺_{β2}B₃³⁺_{β3})₂O₄; and
(A₁²⁺_{α1}A₂²⁺_{α2}A₃²⁺_{α3}A₄²⁺_{α4})·(B₁³⁺_{β1}B₂³⁺_{β2}B₃³⁺_{β3})₂O₄.
wherein the divalent metal A₁²⁺ is selected from the group consisting Ni²⁺, Co²⁺, Cu²⁺ and Zn²⁺, the trivalent metal Bⱼ³⁺ is selected from the group consisting of Fe³⁺, Cr³⁺ and Al³⁺; 0<αi≤1, Σ αi=1, 0<βj≤1, Σ βj=1.

14. A catalyst comprising molybdenum dispersed on the spinel of Formula 1 as defined in claim 13.

15. The catalyst according to claim 14, selected from the group consisting of:
Mo/Ni(Fe_{0.5}Cr_{0.5})₂O₄, Mo/(Ni_{0.5}Cu_{0.5})(Fe_{0.5}Cr_{0.5})₂O₄, Mo/(Ni_{0.5}Co_{0.5})(Fe_{0.5}Cr_{0.5})₂O₄ and Mo/(Ni_{0.5}Zn_{0.5})(Fe_{0.5}Cr_{0.5})₂O₄.

16. A sol-gel method for preparing a spinel of Formula 1 selected from the group consisting of:
(A₁²⁺)·(B₁³⁺_{β1}B₂³⁺_{β2})₂O₄;
(A₁²⁺_{α1}A₂²⁺_{α2})·(B₁³⁺_{β1}B₂³⁺_{β2})₂O₄;
(A₁²⁺_{α1}A₂²⁺_{α2}A₃²⁺_{α3})·(B₁³⁺_{β1}B₂³⁺_{β2})₂O₄;
(A₁²⁺_{α1}A₂²⁺_{α2}A₃²⁺_{α3})·(B₁³⁺_{β1}B₂³⁺_{β2}B₃³⁺_{β3})₂O₄; and
(A₁²⁺_{α1}A₂²⁺_{α2}A₃²⁺_{α3}A₄²⁺_{α4})·(B₁³⁺_{β1}B₂³⁺_{β2}B₃³⁺_{β3})₂O₄.
wherein the divalent metal A₁²⁺ is selected from the group consisting Ni²⁺, Co²⁺, Cu²⁺ and Zn²⁺, the trivalent metal Bⱼ³⁺ is selected from the group consisting of Fe³⁺, Cr³⁺ and Al³⁺; with 0<αi≤1, Σ αi=1, 0<βj≤1, Σ βj=1, the process comprising dissolving in water Ni, Co, Zn, Cu, Fe, Cr and Al salts to form Ni²⁺, Co²⁺, Zn²⁺, Cu²⁺, Fe³⁺, Cr³⁺ and Al³⁺ solution, adding a complexing agent to the salt solution, heating the mixture to 60-90° C. until the gel is formed, and recovering a spinel powder.

17. A process according to claim 16, further comprising the step of loading molybdenum onto the spinel surface by impregnation.

18. An apparatus suitable for converting sour natural gas to sweet natural gas, hydrogen and carbon disulfide, comprising:
a reformer reactor (1), packed with a catalyst, supplied by a feed line [1] from a sour natural gas reservoir, optionally by a feed line [2] connected to an external fresh CO₂ source; and by one or more recycle lines;
a separation unit (S) connected to the outlet of the reformer reactor (1) through a line [4] equipped with a heat exchanger and a compressor; the separation unit consisting of membrane separator(s) (2), gas-liquid separators arranged in series, with a heat exchanger in the line connecting a pair of adjacent gas-liquid separators (3) and a terminus gas-liquid separator (4);
acidic gas removal unit (5), which comprises either an absorption unit filled with a liquid, suitable for separating a gas mixture passing therethrough by dissolving one or more acidic components of the mixture, or a membrane separator; wherein the acidic gas removal unit (5) is supplied by one or more feed lines from the separation unit (S), and is connected by a recycle line [13] to the reformer reactor (1) and through a product delivery line [14] to a WGS reactor (6);
one or more WGS reactors in series (6), wherein the first WGS reactor is supplied with a steam feed line [15] and a feed line [14] that is connected to the outlet of the acidic gas removal unit (5), to deliver one or more gas components which were not captured in the acidic gas removal unit, to said first WGS reactor;
hydrogen separation membrane unit (7), configured to receive a non-condensable component of the effluent of the WGS reactor, or of the last WGS reactor is said series of WGS reactors, wherein the permeate side of said hydrogen separation membrane unit (7) is connected [19 ] to a plant suitable for producing liquid hydrocarbons from hydrogen and CO₂; such that hydrogen and CO₂-containing permeate generated in said membrane can be used as a feed material in production of liquid hydrocarbons in said plant;
optionally a combustion chamber (8), connected [20, 26] to the retentate side of said hydrogen separation membrane unit (7), to receive CH₄-containing stream, wherein the combustion chamber is supplied by an oxygen feed line [27], wherein the combustion chamber is linked to the reformer reactor to supply heat by radiation and convection, and deliver CO₂ combustion product [28] to the inlet of said reactor or as a feed material in production of liquid hydrocarbons in said plant;
optionally an WGS reactor connected [20, 21] to the retentate side of said hydrogen separation membrane unit (7), to receive CH₄ and CO-containing stream, wherein the WGS reactor is supplied with a steam feed line [22], with gas-liquid separator placed downstream of said WGS reactor to recover sweet natural gas [25] and water [24];
wherein the separation unit (S) consists of:
A) a single or multistage membrane separator(s) (2);
B) n gas-liquid separators positioned in series (3₁, . . . , 3ₙ; n≥2; e.g., 3≤n≤7), wherein lines delivering condensates from said gal-liquid separators (3₁, . . . , 3ₙ) are joined to provide a feed line for the terminus gas-liquid separator (4) which is configured to operate under atmospheric pressure, wherein the liquid discharge line [7] of said terminus gas-liquid separator (4) is connected to a storage tank for holding CS₂, with a recycle line [8] connecting the gas outlet of said terminus gas-liquid separator (4) to the reformer reactor (1); wherein
either A is upstream of B, in which case:
the gas-liquid separator (3ₙ) is connected [6] by a pipe to supply non-condensable matter to acidic gas removal unit (5); and
when A) consists of a single membrane separator (2), then the retentate side of said single membrane separator is connected to the inlet of the first gas-liquid separator (3₁), and the permeate side of said single membrane separator (2) is connected to the reformer reactor; or
when A) consists of a multistage membrane separator (2), then the retentate and permeate sides of the first stage membrane separator are connected to the inlet of the first gas-liquid separator (31) and to a second stage membrane separator, respectively, and for any stage other than the first stage, the permeate side is connected by recycle line [10] to the reformer reactor (1) and the retentate side is either connected to the next stage or, in case of the last stage, to an acidic gas removal unit (5) via pipe [11];
or A is downstream to B, in which case then the gas-liquid contactor (3ₙ) is connected by a pipe to supply non-condensable matter to a single or multistage membrane separator(s) (2).
